(19) United States
(12) Patent Application Publication
(10) Pub. No.: US 2007/0276277 A1
(43) Pub. Date: Nov. 29, 2007

(54) Device and method of manual measurement of pulse or respiratory rate

(76) Inventors: John Booth, Tampa, FL (US); Richard Medero, Tampa, FL (US); Donald Brodnick, Cedarburg, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/439,578

(22) Filed: May 24, 2006

(51) Int. Cl. A61B 5/04

(52) U.S. Cl. 600/519

(57) ABSTRACT

An electronic device and method includes a means for setting a time interval for collecting a set of physiological data and entering the number of physiological events that occur within that time interval. The device and method are further configured to calculate the number events that occurred per minute, and displays the results for the user. The device may be implemented as a hand-held device and the method implemented in a graphical user interface of an electronic device.

### FIELD OF THE INVENTION

The invention relates to the field of patient monitoring. More particularly, the invention relates to the field of vital sign monitoring and measurement.

### BACKGROUND OF THE INVENTION

In patient monitoring environments, clinicians manually measure pulse rate by counting the number of pulses during a fixed interval and mentally multiplying the count by a factor equal to 60/interval time. For example, if the clinician sets the time interval at 6 seconds, and counts the number of pulse beats for a patient in any given 6 second interval, the clinician will then multiply the number of heart beats that occurred during that second interval by 10 to come to the heart rate. Likewise, if the clinician sets the interval time to 10 seconds, then the number of heartbeats counted during that 10 second time period will be multiplied by 6.

A similar technique is used for manual measurement of respiration rate. For example, the clinician may count the number of breaths taken by the patient during a 30 second time period and multiply that number of breaths by 2 to come up with a respiration rate in breaths per minute. In both of these instances, the human acts of performing mental math and the timing techniques involved are sources of measurement error. What is needed is an aide to eliminate one or both of these factors.

### SUMMARY OF THE INVENTION

An electronic device and method includes a means for setting a time interval for collecting a set of physiological data and entering the number of physiological events that occur within that time interval. The device and method are further configured to calculate the number events that occurred per minute, and displays the results for the user. The device may be implemented as a hand-held device and the method implemented in a graphical user interface of an electronic device.

In one aspect of the present invention, an electronic device for measuring a physiological event rate of a patient comprises an event register configured to receive a count of a plurality of physiological events over a predetermined time interval, a start button configured to start the predetermined time interval, and a processor configured to calculate the physiological event rate of the patient by multiplying the count of the plurality of physiological events by a factor. The device further comprises a display that is configured to display the predetermined time interval in a count-down or a count-up fashion and further comprises a notification configured to signal the end of the predetermined time interval. The factor is represented by the following equation, factor=60/(the predetermined time interval), wherein the predetermined time interval is measured in seconds. The device further comprises a result indicator, wherein the result indicator is configured to display the physiological event rate. The plurality of physiological events may be a pulsebeat or a breath, and the predetermined time interval is pre-programmed into the processor or is adjustable by a user. The event register receives the count after the predetermined time interval ends, or synchronous with the plurality of physiological events. The event register receives the count from a user or from a sensor. The start button includes voice recognition capabilities, such that a user starts and stops the predetermined time interval, and the event register receives the count with a voice command by the user.

Another aspect of the present invention includes an electronic device for measuring a physiological event rate of a patient comprising a graphical user interface, the graphical user interface configured to allow input from a user, the graphical user interface including an event register configured to receive a count of a plurality of physiological events over a predetermined time interval, a start button configured to start the predetermined time interval, and a processor configured to calculate the physiological event rate of the patient by multiplying the count of the plurality of physiological events by a factor. The graphical user interface further includes a time interval indicator, wherein the time interval indicator is configured to display the predetermined time interval in a count-down or a count-up fashion and further comprises a notification configured to signal the end of the predetermined time interval wherein the factor is represented by the following equation: factor=60/(the predetermined time interval), wherein the predetermined time interval is measured in seconds. The plurality of physiological events in the device are any one of a pulsebeat or a breath. The predetermined time interval is pre-programmed into the processor or may be adjustable by the user and the event register receives the count after the predetermined time interval ends or the count synchronous with the plurality of physiological events. The event register of the present invention may receive the count from the user or a sensor. The event register and the start button include voice recognition capabilities, such that a user starts and stops the predetermined time interval, and the event register receives the count with a voice command by the user.

Yet another aspect of the present invention is a method of measuring a physiological event rate of a patient with an electronic device comprising starting a predetermined time interval, collecting a plurality of physiological events from the patient, recording a count of the plurality of physiological events in the electronic device, and calculating the physiological event rate with a processor in the electronic device by multiplying the count by a factor. The method further comprises setting the predetermined time interval by a user and displaying the physiological event rate on a display. The factor is represented by the following equation: factor=60/(the predetermined time interval), wherein the predetermined time interval is measured in seconds.

Yet another aspect of the present invention is a method of measuring a physiological event rate with an electronic device comprising starting a predetermined time interval, collecting by a user a predetermined number of physiological events from a patient, displaying a calculated physiological event rate for each second of the pre-determined time interval based on the predetermined number of events, and recording the calculated physiological event rate displayed when a last one of the predetermined number of physiological events are collected from the patient.

### BRIEF DESCRIPTION OF THE DRAWINGS

FIG. 1 is a graphical representation of an embodiment of the device of the present invention.

FIG. 2 is a graphical representation of an embodiment of the device of the present invention.

FIG. 3 is a graphical representation of an embodiment of the device and method of the present invention incorporating a graphical user interface.

FIG. 4 is a flow chart of an embodiment of the method of the present invention.

### DETAILED DESCRIPTION

The device as described may include a graphical display including a preset timer that is started coincident with a pulse or respiration event. The timer would countdown from a pre-defined time interval. Several separate embodiments may be implemented, including first where the user enters the number of events that occurred at the end of the time interval, wherein the device is configured to automatically calculate the rate. A second embodiment includes the user tapping a button or making a voice command every time an event occurs during the time interval, wherein the system is configured to automatically calculate the rate. A further embodiment will include a hands off device which will automatically calculate and display a heart rate every second for a pre-set number of counted events.

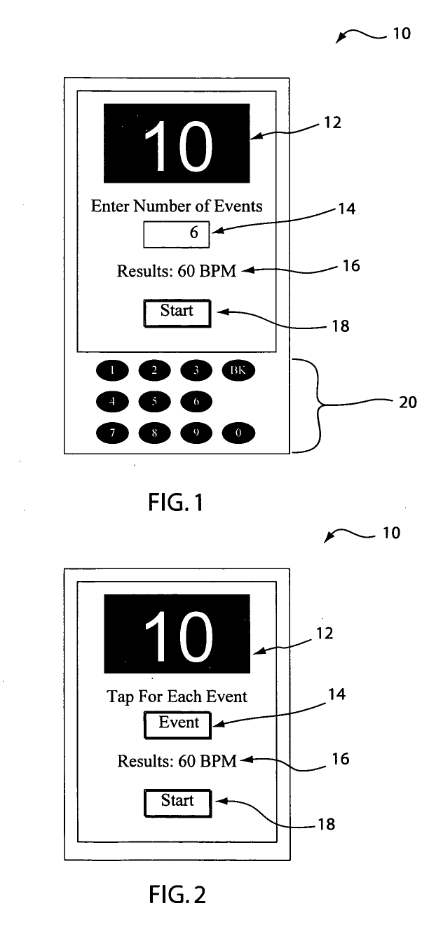

FIG. 1 illustrates an embodiment of the measurement device 10. In this embodiment, the measurement device 10 is a hand-held electronic device that a user may easily hold and manipulate while taking physiological event counts from a patient. In other embodiments, the measurement device 10 may be larger in size, or may be specifically sized to the needs of the medical personnel. The measurement device 10 includes an interval display 12, that displays the length of the time interval in seconds used to collect the physiological events from the patient, and further counts up to or down from a predetermined time interval. One embodiment, the time interval shown on an interval display 12 is preset and cannot be changed, however, additional embodiments may include the ability to set the time interval in the interval display 12 by a user.

Still referring to FIG. 1, the measurement device 10 also includes an event register 14. The event register 14 is utilized to enter the amount of physiological events that occurred during the time interval. In the embodiment shown in FIG. 1, the number of physiological events are entered into the event register 14 after the time interval has ended. The number of physiological events may be entered into the event register 14 using the data entry buttons 20. In alternative embodiments, the data entry buttons 20 may also be used to change the time interval shown in the interval display 12. The start button 18 of the measurement device 10 in FIG. 1 is activated by a user, and starts the time interval as shown in the interval display 12. Finally, the result indicator 16 displays the final calculation of the physiological event rate in events per minute.

In operation, the predefined interval of the measurement device 10 is set and displayed in the interval display 12. As stated previously, one embodiment will include a measurement device 10 having a pre-programmed interval, while other embodiments will include the ability for a user to adjust the predetermined time interval. Once the user is ready to collect a set of physiological events from the patient, the user presses the start button 18, and the time interval begins to count down or count up. During the time interval, the user collects the set of physiological events from the patient, and at the end of the time interval, enters the number of events in the event register 14 using the data entry buttons 20. In one embodiment, an alarm will indicate when the time interval is over. Once the user has entered the number of events in the event register 14, the measurement device 10 will calculate the physiological event rate and display it in the result indicator 16.

FIG. 2 illustrates an embodiment of the measurement device 10. Here, the measurement device 10 also includes an interval display 12, a start button 18, and a result indicator 16 as shown above in FIG. 1. The event register 14 of the measurement device 10 shown in FIG. 2 requires the user to enter each physiological event synchronistic with the event occurring. In other words, the user of the measurement device 10 in FIG. 2 will activate the event register 14 every time a physiological event takes place, such as touching the event register 14 every time a pulse beat occurs in the patient. Once the predetermined time interval has ended, the measurement device 10 will automatically calculate the physiological event rate and display it in the result indicator 16. It is further contemplated that the measurement device 10 may also include a sensor (not shown) that would collect and enter into the event register 14 each physiological event, such that the user would not need to synchronically activate the event register 14 every time a physiological event took place. Such a sensor could also be implemented on the measurement device 10 as depicted in FIG. 1 as well. A further embodiment will also implement voice recognition capabilities, such that a user of the measurement device 10 may speak to the measurement device 10 a command to start the countdown, and further record every event by speaking to the measurement device 10. Also, the user will be able to ask the measurement device 10 to display the heart rate in the result indicator 16. Preferably, the user would use the word “start” to start the countdown, the word “beat” or “breath” to record every event, and the word “rate” to prompt the measurement device 10 to display the rate in the result indicator 16. It should be noted that the interval display 12, the event register 14, the result indicator 16, the start button 18, as well as the data entry buttons 20 may be configured in any way, and in any order that is convenient on the measurement device 10.

In yet another embodiment, a complete “hands off” device is contemplated. Referring again to FIG. 2, this measurement device 10 would be activated either by using the start button 18 for voice recognition as was discussed previously. The measurement device 10 would be preprogrammed with a time cycle, for example, a 20 second cycle. In use, the user would find the pulse or breath of the patient and wait for the interval display 12 for instruction. The interval display 12 displays a static message such as “count 10 heartbeats”, and then starts the 20 second cycle with the message “begin.” At this point, the user would start counting the patient events, up to a predetermined number of events, in this case 10. At various points throughout the 20 second cycle, perhaps starting at one second and showing every second, or perhaps starting at the third second, and showing every second the interval display 12 will display an event rate representing the rate of the patient's event if the 10^{th} event were recorded at that time. For example, in the first second, the interval display 12 would display 600 bpm, with would represent the patient's heart rate if 10 heartbeats were recorded in the first second. Likewise, in the 2^{nd} second, 300 bpm will be displayed in the interval display 12, representing a patient's heart rate if 10 beats were counted in 2 seconds. As a final example, in the 20^{th} second, the interval display will read 30 bpm, representing a patient's heart rate with 10 beats are counted in the 20 second cycle. A user will start the 20 second cycle and be able to view the approximate heart rate of the patient when the user counts the 10^{th} beat. At the end of the 20 second cycle, the word “begin” will reappear in the interval display 12, and the user may once again start count 10 beats. It should be noted that alternative embodiments will include the ability to set the time cycle, as well as the number of beats being counted.

It is also contemplated that this aforementioned embodiment may be used to count breaths of the patient as well. However, in this embodiment, the number of breaths counted will likely be in a range from 3-5 in the given time cycle. However, the principals described above in the illustration of the heartbeat embodiment would likewise apply to the counting of the patient's breaths. The one difference being that the user of the measurement device 10 must gauge where in a breath cycle the count starts and observe the rate at that point in the next breath cycle.

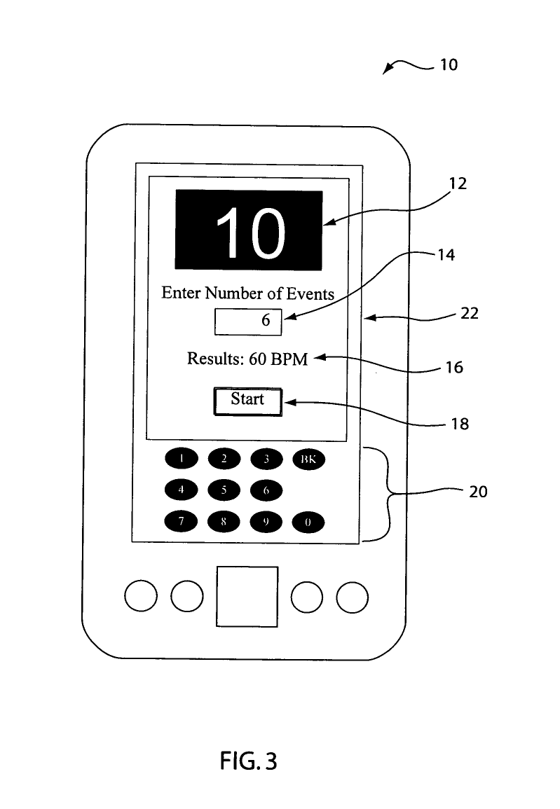

An additional embodiment of the measurement device 10 is illustrated in FIG. 3. Here, the measurement device 10 is a device such as a PDA, laptop, or some other electronic device having a graphical user interface 22 with touch screen capabilities. In such an embodiment, a user may utilize a stylus (not shown) or their finger, or some other tool to enter the appropriate number of physiological events, start the interval, or set the time interval by touching the graphical user interface 22. This measurement device 10 may utilize either the configuration from FIG. 1 or the configuration from FIG. 2 as the operating interface to be displayed on the graphical user interface 22. Likewise, such a measurement device 10 may also utilize a sensor (not shown) to collect the number of physiological events.

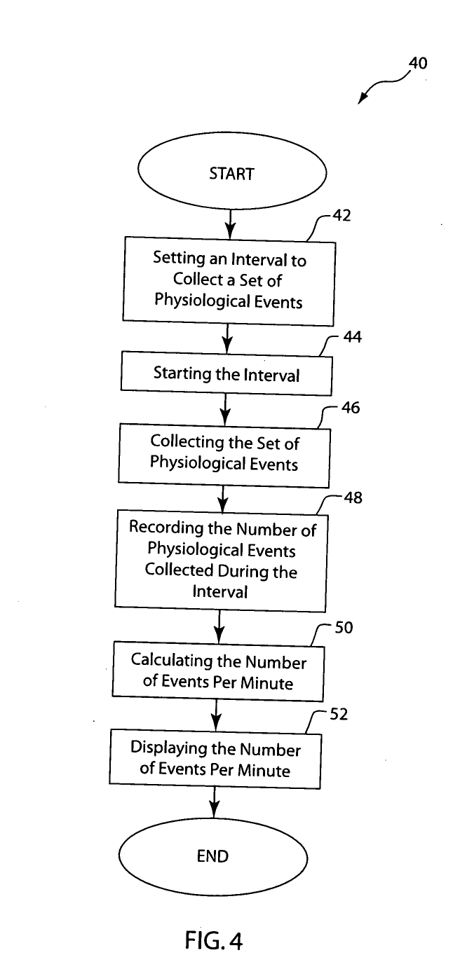

An embodiment of a measurement method 40 is depicted in FIG. 4. In step 42, an interval is set to collect a set of physiological events. As stated previously, the interval may be set and pre-programmed in a measurement device, or may be adjustable by a user. In step 44, the interval is started by the user, and in step 46 the set of physiological events is collected by the user. As stated previously, the physiological events in step 46 may also be collected by a sensor.

Still referring to FIG. 4, in step 48, the number of physiological events collected during the interval are recorded and entered into the measurement device. As stated previously, the physiological events may be recorded after the end of the time interval, or synchronously with the occurrence of the physiological events. Once the number of physiological events are recorded and entered in the measurement device in step 48, the number of physiological events per minute are calculated in step 50 by the electronic device. And in step 52 the number of events per minute are displayed for the user.

This device and method have a number of advantages over the prior art, in that accuracy of the measurement of pulse rate and respiratory rate would be vastly improved, and no mental arithmetic would be required by the clinician. Furthermore, patient data resulting from the measurement can be easily captured in an electronic medical record.

The present invention has been described in terms of specific embodiments incorporating details to facilitate the understanding of the principals of construction and operation of the invention. Such reference herein to specific embodiments and details thereof is not intended to limit the scope of the claims appended hereto. It will be apparent to those skilled in the art that modifications may be made in the embodiment chosen for illustration without departing from the spirit and scope of the invention.

What is claimed is:

1. An electronic device for measuring a physiological event rate of a patient, the electronic device comprising:
an event register configured to receive a count of a plurality of physiological events over a predetermined time interval;
a start button configured to start the predetermined time interval; and
a processor configured to calculate the physiological event rate of the patient by multiplying the count of the plurality of physiological events by a factor.

2. The device as claimed in claim 1, further comprising a display, wherein the display is configured to display the predetermined time interval in a count-down or a count-up fashion.

3. The device as claimed in claim 1, further comprising a notification configured to signal the end of the predetermined time interval.

4. The device as claimed in claim 1, wherein the factor is represented by the following equation:
factor=60/the predetermined time interval,
wherein the predetermined time interval is measured in seconds.

5. The device as claimed in claim 1, further comprising a result indicator, wherein the result indicator is configured to display the physiological event rate.

6. The device as claimed in claim 1, wherein the plurality of physiological events are any one of the following:
a pulsebeat; and
a breath.

7. The device as claimed in claim 1, wherein the predetermined time interval is pre-programmed into the processor.

8. The device as claimed in claim 1, wherein the predetermined time interval is adjustable by a user.

9. The device as claimed in claim 1, wherein the event register receives the count after the predetermined time interval ends.

10. The device as claimed in claim 1, wherein the event register receives the count synchronous with the plurality of physiological events.

11. The device as claimed in claim 1, wherein the event register receives the count from a user.

12. The device as claimed in claim 1, wherein the event register receives the count from a sensor.

13. The device as claimed in claim 1, wherein the event register and the start button include voice recognition capabilities, such that a user starts and stops the predetermined time interval, and the event register receives the count with a voice command by the user.

14. An electronic device for measuring a physiological event rate of a patient, the electronic device comprising:
a graphical user interface, the graphical user interface configured to allow input from a user, the graphical user interface including:
an event register configured to receive a count of a plurality of physiological events over a predetermined time interval; and
a start button configured to start the predetermined time interval; and
a processor configured to calculate the physiological event rate of the patient multiplying the count of the plurality of physiological events by a factor.

15. The device as claimed in claim 14, wherein the graphical user interface further includes a time interval indicator, wherein the time interval indicator is configured to display the predetermined time interval in a count-down or a count-up fashion.

16. The device as claimed in claim 14, further comprising a notification configured to signal the end of the predetermined time interval.

17. The device as claimed in claim 14, wherein the factor is represented by the following equation:
factor=60/the predetermined time interval,
wherein the predetermined time interval is measured in seconds.

18. The device as claimed in claim 14, wherein the plurality of physiological events are any one of the following:
a pulsebeat; and
a breath.

19. The device as claimed in claim 14, wherein the predetermined time interval is pre-programmed into the processor.

20. The device as claimed in claim 14, wherein the predetermined time interval is adjustable by the user.

21. The device as claimed in claim 14, wherein the event register receives the count after the predetermined time interval ends.

22. The device as claimed in claim 14, wherein the event register receives the count synchronous with the plurality of physiological events.

23. The device as claimed in claim 14, wherein the event register receives the count from the user.

24. The device as claimed in claim 14, wherein the event register receives the count from a sensor.

25. The device as claimed in claim 14, wherein the event register and the start button include voice recognition capabilities, such that a user starts and stops the predetermined time interval, and the event register receives the count with a voice command by the user.

26. A method of measuring a physiological event rate of a patient with an electronic device, the method comprising:
starting a predetermined time interval;
collecting a plurality of physiological events from the patient;
recording a count of the plurality of physiological events in the electronic device; and
calculating the physiological event rate with a processor in the electronic device by multiplying the count by a factor.

27. The method as claimed in claim 26, further comprising setting the predetermined time interval by a user.

28. The method as claimed in claim 26, further comprising displaying the physiological event rate on a display.

29. The method as claimed in claim 26, wherein the factor is represented by the following equation:
factor=60/the predetermined time interval
wherein the predetermined time interval is measured in seconds.

30. A method of measuring a physiological event rate with an electronic device, the method comprising:
starting a predetermined time interval;
collecting by a user a predetermined number of physiological events from a patient;
displaying a calculated physiological event rate for each second of the pre-determined time interval based on the predetermined number of events; and
recording the calculated physiological event rate displayed when a last one of the predetermined number of physiological events are collected from the patient.
